(19) United States
(12) Patent Application Publication
(10) Pub. No.: US 2023/0058772 A1
(43) Pub. Date: Feb. 23, 2023

(54) DISPOSABLE ENDOSCOPE SHEATH

(71) Applicant: Nizam M. MEAH, Manvel, TX (US)

(72) Inventor: Nizam M. MEAH, Manvel, TX (US)

(21) Appl. No.: 17/406,628

(22) Filed: Aug. 19, 2021

(51) Int. Cl. A61B 1/00

(52) U.S. Cl. CPC A61B 1/00135; A61B 1/00137

(57) ABSTRACT

Embodiments of the present disclosure provide a disposable sheath for an endoscope device. The endoscope sheath includes an elongated conduit including a first end and a second end. The elongated conduit is configured with a plurality of hollow ports extending from a proximal portion to a distal portion of the elongated conduit. Further, the sheath includes a shield body member including a first flap member and a second flap member extending from the first end and the second end, respectively. The first flap member and the second flap member are operated between a closed position and an open position for encasing the endoscope device for performing an endoscopy. The sheath includes an endoscope cap adapted to releasably engage with a portion of the elongated conduit and the shield body member encompassing a distal end of the endoscope device.

### TECHNICAL FIELD

The present disclosure relates to medical devices and, more particularly relates, to a disposable sheath for an endoscope device.

### BACKGROUND

In the recent past, one of the common diagnostic procedures employed by medical practitioners for examining an internal organ (e.g., a stomach) of a subject is endoscopy. Endoscopy involves the insertion of an endoscope into the internal organ of the subject for visual inspection and thereafter a suitable medication is prescribed. Endoscopy has proliferated recently due to its non-invasive nature and the ability of the medical practitioner to access intricate areas of the internal organ for diagnosis.

However, an endoscope device in direct contact with the tissues of the internal organ causes several potential health risks. In one scenario, maneuvering the endoscope within the internal organ may cause bleeding at the tissue (e.g., perforation at a gut wall) due to force exerted by the internal organ on a rigid body of the endoscope. Further, bleeding at the tissue may also lead to infections. Moreover, due to the usage of the same endoscope device for performing endoscopy for multiple patients, the endoscope device may act as an infection carrier from one patient to another. Conventionally, to prevent the spread of the infection, the endoscope is mainly washed and disinfected before each use. Although, the entire endoscope can be washed and disinfected to avoid such contamination, however, this may require considerable time and effort, which may adversely affect the working efficiency of the endoscope.

Therefore, there is a need for a disposable sheath and/or cover for the endoscopes in addition to providing other technical advantages.

### SUMMARY

Various embodiments of the present disclosure provide a disposable sheath for an endoscope device.

In an embodiment, a disposable sheath for an endoscope device is disclosed. The disposable sheath includes an elongated conduit. The elongated conduit includes a first end and a second end. The elongated conduit is configured with a plurality of hollow ports extending from a proximal portion to a distal portion of the elongated conduit. The disposable sheath includes a shield body member. The shield body member includes a first flap member and a second flap member extending from the first end and the second end, respectively. The first flap member and the second flap member are configured along a length of the elongated conduit. The first flap member and the second flap member are operated between a closed position and an open position for encasing the endoscope device for performing an endoscopy. The disposable sheath includes an endoscope cap adapted to releasably engage with a portion of the elongated conduit and the shield body member encompassing a distal end of the endoscope device.

In another embodiment, a disposable sheath for an endoscope device is disclosed. The disposable sheath includes an elongated conduit. The elongated conduit includes a plurality of hollow ports extending from a proximal portion to a distal portion of the elongated conduit. Further, the elongated conduit includes a first socket member and a second socket member configured at a first end and a second end, respectively of the elongated conduit. The first socket member and the second socket member extend from the proximal portion to the distal portion. The disposable sheath further includes a shield body member. The shield body member includes a primary end and a secondary end. The primary end and the secondary end are configured with a first engagement member and a second engagement member, respectively. The shield body member is configured to operate between an engaged position and a disengaged position for encasing the endoscope device. The disposable sheath includes an endoscope cap adapted to releasably engage with a portion of the elongated conduit and the shield body member encompassing a distal end of the endo scope device.

### BRIEF DESCRIPTION OF THE FIGURES

The following detailed description of illustrative embodiments is better understood when read in conjunction with the appended drawings. For the purpose of illustrating the present disclosure, exemplary constructions of the disclosure are shown in the drawings. However, the present disclosure is not limited to a specific device or a tool and instrumentalities disclosed herein. Moreover, those in the art will understand that the drawings are not to scale. Wherever possible, like elements have been indicated by identical numbers:

FIG. 1A illustrates a schematic view of a disposable sheath for an endoscope device, in accordance with an example embodiment of the present disclosure;

FIG. 1B is a sectional view of a portion of the sheath of FIG. 1A, depicting the sheath operated in an open position, in accordance with an example embodiment of the present disclosure;

FIG. 1C is a sectional view of a portion of the sheath of FIG. 1A, depicting the sheath operated in a closed position, in accordance with an example embodiment of the present disclosure;

FIG. 1D is a sectional view of a portion of the sheath of FIG. 1A, depicting an endoscope cap attached to a portion of an elongated conduit and a shield body member covering the endoscope device, in accordance with an example embodiment of the present disclosure;

FIG. 1E is a cross-sectional view of a portion of the sheath of FIG. 1D, in accordance with an example embodiment of the present disclosure;

FIGS. 2A-2B illustrate sectional views of a portion of the sheath of FIG. 1A, in accordance with an example embodiment of the present disclosure;

FIG. 3A illustrates a schematic view of a sheath for a medical device, in accordance with another example embodiment of the present disclosure;

FIG. 3B is a sectional view of a portion of the sheath of FIG. 3A, depicting the sheath operated in an disengaged position, in accordance with an example embodiment of the present disclosure;

FIG. 3C is a sectional view of a portion of the sheath of FIG. 3A, depicting the sheath operated in an engaged position, in accordance with an example embodiment of the present disclosure;

FIG. 3D is a sectional view of a portion of the sheath of FIG. 3A, depicting an endoscope cap attached to a portion of an elongated conduit and a shield body member covering the endoscope device, in accordance with an example embodiment of the present disclosure;

FIG. 3E is a cross-sectional view of a portion of the sheath of FIG. 3D, in accordance with an example embodiment of the present disclosure;

FIGS. 4A-4B illustrate sectional views of a portion of the sheath of FIG. 3A, in accordance with an example embodiment of the present disclosure; and

FIG. 5 is a schematic representation of the sheath arranged on the endoscope and inserted to an internal organ of a subject, in accordance with an embodiment of the present disclosure.

The drawings referred to in this description are not to be understood as being drawn to scale except if specifically noted, and such drawings are only exemplary in nature.

### DETAILED DESCRIPTION

In the following description, for purposes of explanation, numerous specific details are set forth in order to provide a thorough understanding of the present disclosure. It will be apparent, however, to one skilled in the art that the present disclosure can be practiced without these specific details. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

Reference in this specification to “one embodiment” or “an embodiment” means that a particular feature, structure, or characteristic described in connection with the embodiment is included in at least one embodiment of the present disclosure. The appearances of the phrase “in an embodiment” in various places in the specification are not necessarily all referring to the same embodiment, nor are separate or alternative embodiments mutually exclusive of other embodiments. Moreover, various features are described which may be exhibited by some embodiments and not by others. Similarly, various requirements are described which may be requirements for some embodiments but not for other embodiments.

Moreover, although the following description contains many specifics for the purposes of illustration, anyone skilled in the art will appreciate that many variations and/or alterations to said details are within the scope of the present disclosure. Similarly, although many of the features of the present disclosure are described in terms of each other, or in conjunction with each other, one skilled in the art will appreciate that many of these features can be provided independently of other features. Accordingly, this description of the present disclosure is set forth without any loss of generality to, and without imposing limitations upon, the present disclosure.

The term ‘endoscope’ used throughout the present disclosure refers to the endoscope device, which is configured with a slender and tubular instrument used for visual diagnostics of internal organs of a subject.

The term ‘endoscopy’ used throughout the present disclosure refers to a diagnostics procedure employed by medical practitioners by using the endoscope.

### Overview

Various embodiments of the present disclosure provide an endoscope sheath. In an embodiment, the sheath includes an elongated conduit and a shield body member. The elongated conduit is configured with a plurality of hollow ports extending from a proximal portion to a distal portion of the elongated conduit. The plurality of hollow ports includes a first hollow port and a second hollow port. The first hollow port is located proximate to a first end and a second end of the elongated conduit and is configured to allow passage of fluids therethrough. The second hollow port is located at a central portion of the elongated conduit and is configured to allow passage for biopsy medical instruments therethrough. Further, the shield body member includes a first flap member and a second flap member extending from the first end and the second end, respectively. The first flap member and the second flap member are operated between a closed position and an open position for encasing the endoscope device for performing an endoscopy. More specifically, the elongated conduit is positioned over the endoscope device and a distal end of each of the first and second flap members is positioned apart from each other in the open position. Thereafter, the distal end of each of the first and second flap members is sealingly coupled with each other by wrapping the first flap member and the second flap member around the endoscope device. In another configuration, the distal end of each of the first flap member and the second flap member may be configured with a first taper surface and a second taper surface, respectively. Thus, in the closed position, the first taper surface is sealingly coupled to the second taper surface by overlapping the first taper surface over the second taper surface upon wrapping the first and second flap members around the endoscope. Further, the sheath includes an endoscope cap adapted to releasably engage with a portion of the elongated conduit and the shield body member encompassing a distal end of the endoscope device.

In an embodiment, the sheath includes an elongated conduit and a shield body member. The elongated conduit is configured with a plurality of hollow ports extending from a proximal portion to a distal portion of the elongated conduit. The elongated conduit further includes a first socket member and a second socket member configured at a first end and a second end of the elongated conduit respectively. Further, the shield body member includes a primary end and a secondary end. The primary end and the secondary end include a first engagement member and a second engagement member, respectively. The shield body member is configured to operate between an engaged position and a disengaged position for encasing the endoscope device. More specifically, the elongated conduit is positioned over the endoscope and the first engagement member and the second engagement member are sealingly coupled with the first socket member and the second socket member, respectively in the engaged position. Further, in the disengaged position, the first engagement member and the second engagement member are disengaged from the first socket member and the second socket member, respectively, thereby splitting the shield body member and the elongated conduit. In another configuration, the first engagement member and the second engagement member may be configured on an exposed surface of the shield body member.

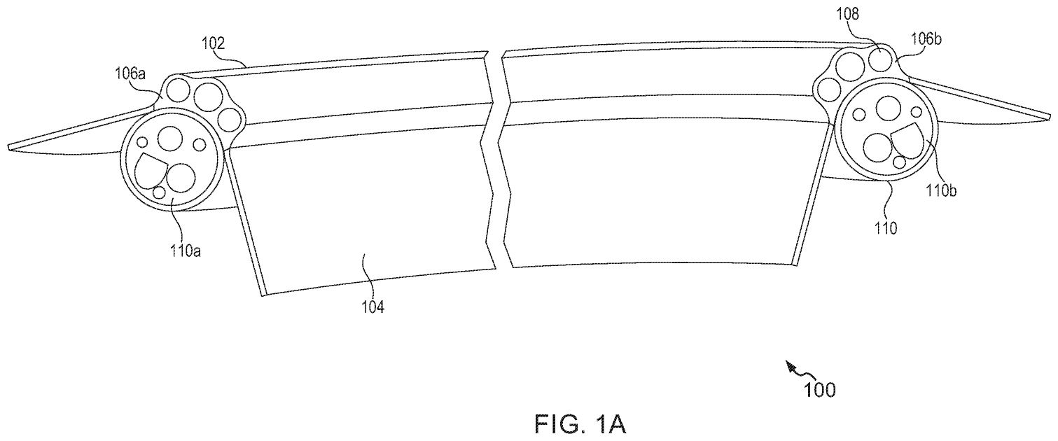

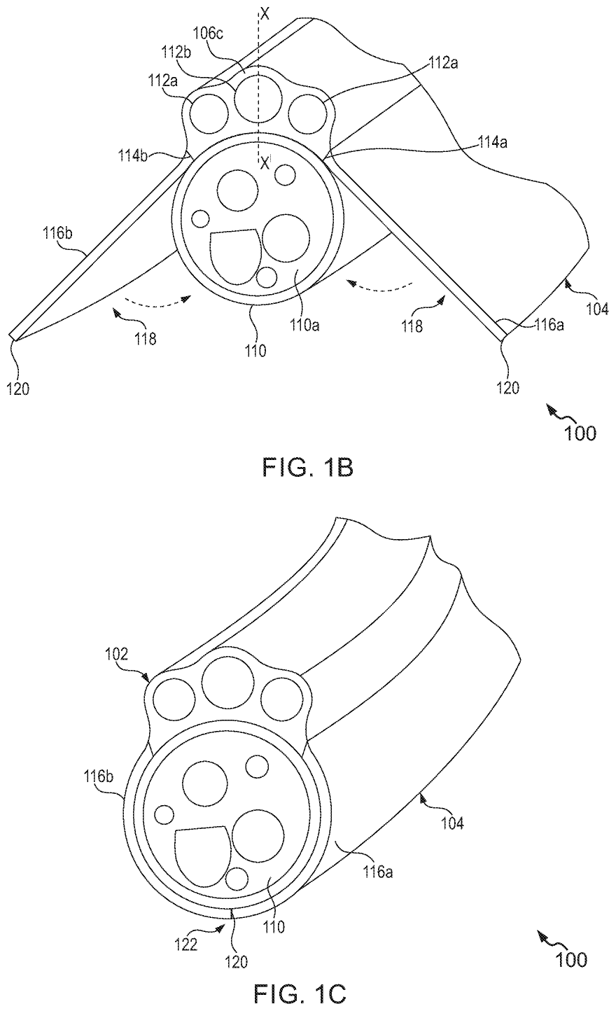

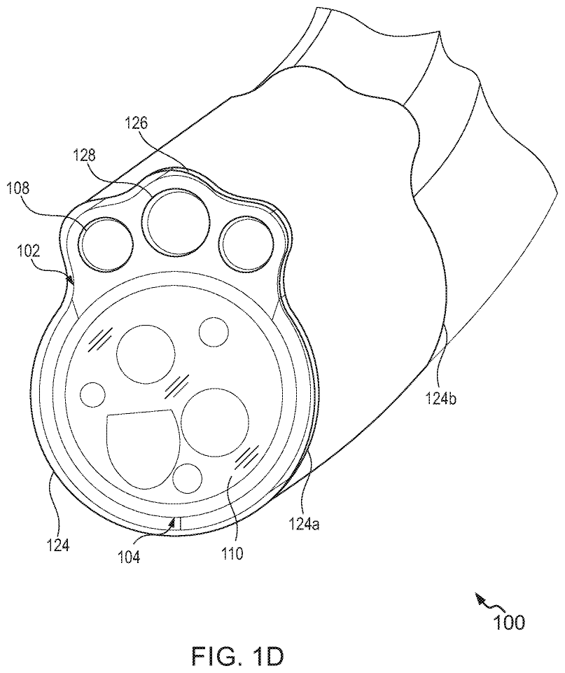

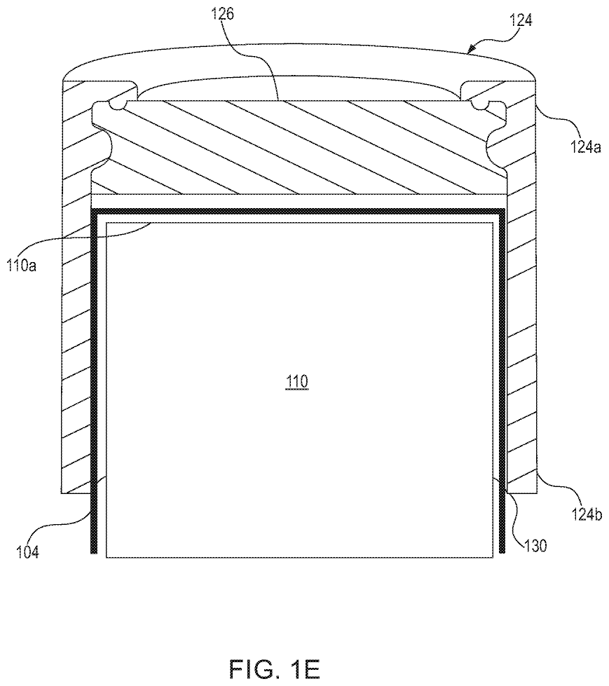

Various embodiments of a disposable endoscope sheath are described with reference to FIGS. 1A-1E to FIG. 5.

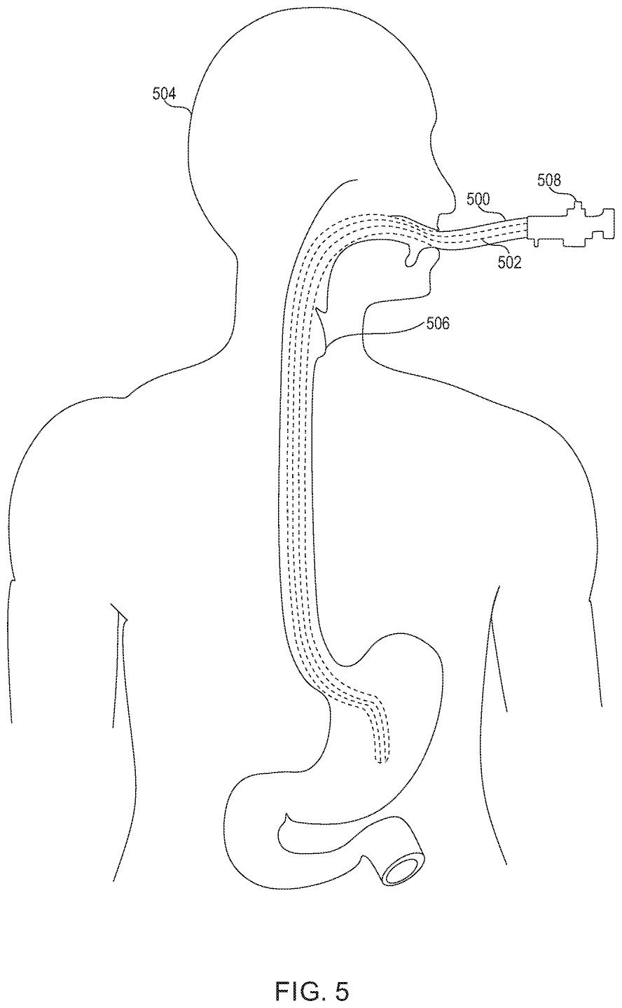

FIG. 1A illustrates a schematic view of a disposable sheath 100 for a medical device, in accordance with an example embodiment of the present disclosure. The disposable sheath 100 (hereinafter interchangeably referred to as “sheath 100”) is configured to encase a medical device 110. As an example, the medical device 110 may be an endoscope device 110 (hereinafter interchangeably referred to as “endoscope 110”) that is used for visual inspection of an internal organ of a subject. The sheath 100 encasing the endoscope 110 is configured to be inserted to the internal organ of the subject during the endoscopy which will be explained with reference to FIG. 5. It should be appreciated that a distal end 110a of the endoscope 110 may be located within the internal organ and a proximal end 110b of the endoscope 110 may be located outside the subject.

The sheath 100 includes an elongated conduit 102 (hereinafter interchangeably referred to as “conduit 102”) and a shield body member 104. The elongated conduit 102 includes a proximal portion 106a and a distal portion 106b. Further, the elongated conduit 102 is configured with a plurality of hollow ports 108 extending from the proximal portion 106a to the distal portion 106b. In other words, the hollow ports 108 are configured along a length of the elongated conduit 102. The hollow ports 108 include a first hollow port 112a and a second hollow port 112b (as shown in FIG. 1B). The first hollow port 112a is configured proximate to a first end 114a and a second end 114b of the elongated conduit 102 and the second hollow port 112b is configured at a central portion 106c of the elongated conduit 102 (as shown in FIG. 1B). The elongated conduit 102 including the first hollow port 112a and the second hollow port 112b is symmetric to an axis X-X′ of the elongated conduit 102 (as shown in FIG. 1B). The first hollow port 112a located proximate to the ends 114a, 114b is configured to allow passage for fluids (i.e. air or water) during the endoscopy. The second hollow port 112b corresponds to a biopsy channel. The biopsy channel allows a medical practitioner to introduce biopsy medical instruments (such as, a biopsy forceps or a thin caliber cannula) during the endoscopy. In an embodiment, the second hollow port 112b may be a vacuum channel for allowing the vacuum therethrough for enhancing visualization of the internal organ while maneuvering the endoscope device 110. Further, the axis X-X′ may be a central axis of the elongated conduit 102. In one configuration, the axis X-X′ may be any axis defined on the elongated conduit 102, configured to define the orientation or position of the first hollow port 112a and the second hollow port 112b configured in the conduit 102, as per design feasibility and requirement. For illustrative purposes, only 3 hollow ports are illustrated in the elongated conduit 102, and they can be in any other number depending on design feasibility and requirement.

It should be noted that the elongated conduit 102 towards the first end 114a (i.e. towards the right side in FIG. 1B) and the second end 114b (i.e. towards the left side in FIG. 1B) from the central axis X-X′ conforms to a stepped profile (as shown in FIG. 1B). The conduit 102 may be made of flexible materials such as, but not limited to, silicon, thermoplastic elastomers (TPE) and the like.

Further, the shield body member 104 includes a first flap member 116a and a second flap member 116b extending from the first end 114a and the second end 114b, respectively. More specifically, the flap members 116a, 116b extend laterally from the first end 114a and the second end 114b, respectively. It is noted that the flap members 116a, 116b are configured along a length of the elongated conduit 102 (i.e. extending the proximal portion 106a to the distal portion 106b) (as shown in FIG. 1A). The flap members 116a, 116b extending from the conduit 102 correspond to integrated components of the sheath 100. The flap members 116a, 116b integrated to the conduit 102 may be defined with a predefined thickness (i.e. uniform thickness) as per the shielding requirements. In one configuration, the thickness of the shield body member 104 may be in the range of about 5 mm to 1 cm, as per feasibility and requirements. In another configuration, the flap members 116a, 116b may be configured with non-uniform thickness. As an example, an outer surface of the flap members 116a, 116b may be configured with a curved surface. In an embodiment, a length dimension of the flap members 116a, 116b may be configured to be equal (as shown in FIG. 1B). In another embodiment, the flap members 116a, 116b may be configured with non-uniform length dimension.

The flap members 116a, 116b may be configured with flexible materials such as, but not limited to, thermoplastic elastomers (TPE), polymers, silicon and the like. The flap members 116a, 116b may be configured to be of rectangular shape with adequate dimensions for wrapping the endoscope 110. In one configuration, the outer surface of the flap members 116a, 116b and the outer surface of the conduit 102 may be provided with a biocompatible lubricant such as TriboSys™ PFPE (perfluoropolyether) or any other biocompatible lubricants, as per the design feasibility and requirements. This configuration eases maneuvering of the endoscope 110 encased within the sheath 100 for accessing the internal organ.

Prior to the endoscopy, the sheath 100 is operated between an open position (see, 118 of FIG. 1B) and a closed position (see, 122 of FIG. 1C) for encasing the endoscope 110. More specifically, the conduit 102 is rested on a portion of an outer surface of the endoscope 110. In this scenario, the flap members 116a, 116b are positioned apart from each other (i.e. positioned on either side of the axis X-X′) (as shown in FIG. 1B). The flap members 116a, 116b positioned apart from each other correspond to the open position 118. Thereafter, the flap members 116a, 116b are operated to the closed position 122 by wrapping the flap members 116a, 116b around the endoscope 110 (as shown in FIG. 1C), thereby encasing the endoscope 110. More specifically, a distal end 120 of each of the first flap member 116a and the second flap member 116b is sealingly coupled with each other by wrapping the first flap member 116a and the second flap member 116b around the endoscope 110 (as shown in FIG. 1C). The distal end 120 of the flap members 116a, 116b includes a biocompatible adhesive for allowing sealing of the flap members 116a, 116b. In one configuration, the biocompatible adhesive may provide temporary adhesion. The adhesion provides adequate coupling and prevents deformation/misalignment of the flap members 116a, 116b while maneuvering the endoscope 110 wrapped with the sheath 100 in the internal organ. Further, the sheath 100 may be disengaged or operated to the open position 118, upon completion of the endoscopy for disposal. In one scenario, the sheath 100 may be washed and re-used.

In an embodiment, the sheath 100 may be dimensioned based on the circumference of the endoscope 110. In this configuration, the sheath 100 may be operated to the closed position 122, thereby forming an opening for allowing the endoscope 110 to be inserted therethrough. The opening formed by the sheath 100 when operated in the closed position 122 conforms to the endoscope 110. In other words, the shape of the opening is configured to be identical to the shape of the outer surface of the endoscope 110.

Further, an endoscope cap 124 is releasably engaged with a portion of the elongated conduit 102 and the shield body member 104 covering the distal end 110a of the endoscope 110 (as shown in FIG. 1D). The endoscope cap 124 facilitates optimal visual field by maintaining the distance between endoscope 110 and the target area (i.e. the internal organ).

The endoscope cap 124 includes a transparent lens 126 (as shown in FIG. 1D). The transparent lens 126 is disposed at a front portion 124a of the endoscope cap 124, thereby forming a closed structure at the front portion 124a of the endoscope cap 124 (as shown in FIGS. 1D and 1E). The transparent lens 126 allows visualization of the internal organ of a subject during the endoscopy which is explained further in detail. Further, the transparent lens 126 is configured with a plurality of orifices 128. The orifices 128 are positioned coaxially with the hollow ports 108 when the endoscope cap 124 is releasably engaged with the portion of the elongated conduit 102 and the shield body member 104 encompassing the distal end 110a of the endoscope 110 (as shown in FIG. 1D). It should be understood that the fluids and the medical instruments passing through the each of the hollow ports 108 enter the internal organ by passing through the orifices 128.

The endoscope cap 124 further includes an aperture 130 configured at a rear portion 124b of the endoscope cap 124 (as shown in FIG. 1E). The aperture 130 is configured to receive the portion of the elongated conduit 102 and the shield body member 104 encompassing the distal end 110a of the endoscope device 110 (as shown in FIG. 1E). The endoscope cap 124 may be configured with dimensions in conformity with the dimensions and shape of the elongated conduit 102 and the shield body member 104 encompassing the endoscope 110. In other words, the endoscope cap 124 conforms to a shape of the elongated conduit 102 and the shield body member 104 encasing the endoscope 110 (as shown in FIG. 1D). This enables the endoscope cap 124 to be snuggly mounted to the portion of the elongated conduit 102 and the shield body member 104 encasing the distal end 110a of the endoscope 110. This prevents misalignment or deformation of the endoscope cap 124 during the endoscopy. In an embodiment, the biocompatible adhesive may be used for temporary sealing of the endoscope cap 124 to the conduit 102 and the shield body member 104 covering the endoscope 110.

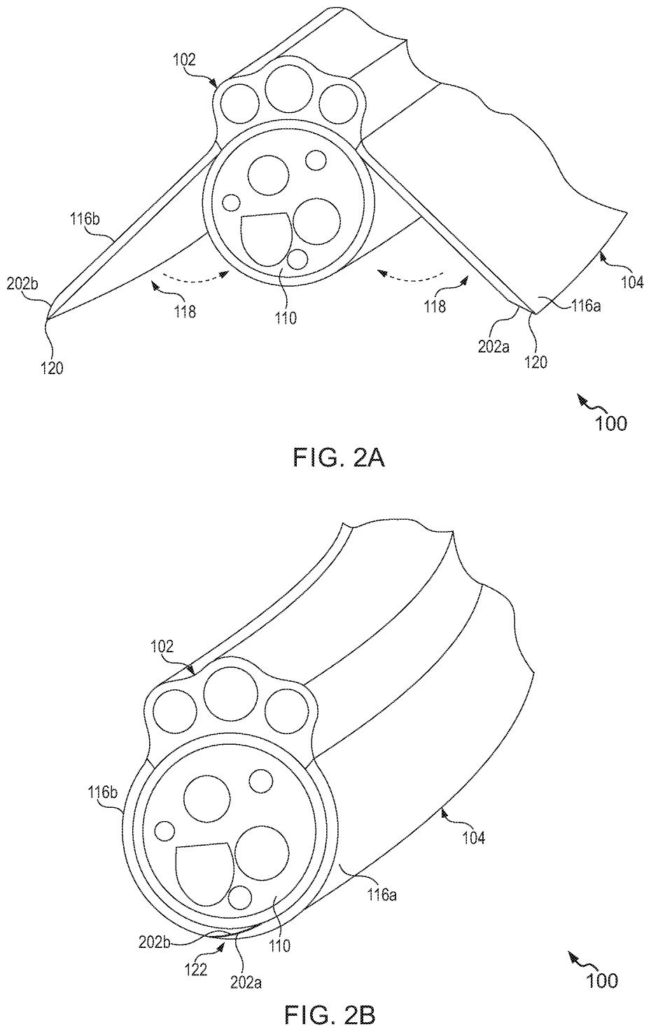

Referring to FIG. 2A, the distal end 120 of each of the flap members 116a, 116b may be configured with a first taper surface 202a and a second taper surface 202b, respectively. The first taper surface 202a corresponds to an inner taper and the second taper surface 202b corresponds to an outer taper. Alternatively, the first taper surface 202a may be configured at the distal end 120 of the second flap member 116b and the second taper surface 202b may be configured at the distal end 120 of the first flap member 116a. The first taper surface 202a is sealingly coupled to the second taper surface 202b by operating the flap members 116a, 116b to the closed position 122 from the open position 118 (as shown in FIG. 2B). More specifically, the first taper surface 202a is overlapped over the second taper surface 202b upon wrapping the flap members 116a, 116b around the endoscope 110 and is sealingly coupled to each other, thereby encasing the endoscope 110 (as shown in FIG. 2B). In this configuration, the endoscope cap 124 is releasably engaged with the portion of the conduit 102 and the shield body member 104 covering the distal end 110a of the endoscope 110, upon wrapping the flap member 116a and 116b as explained above.

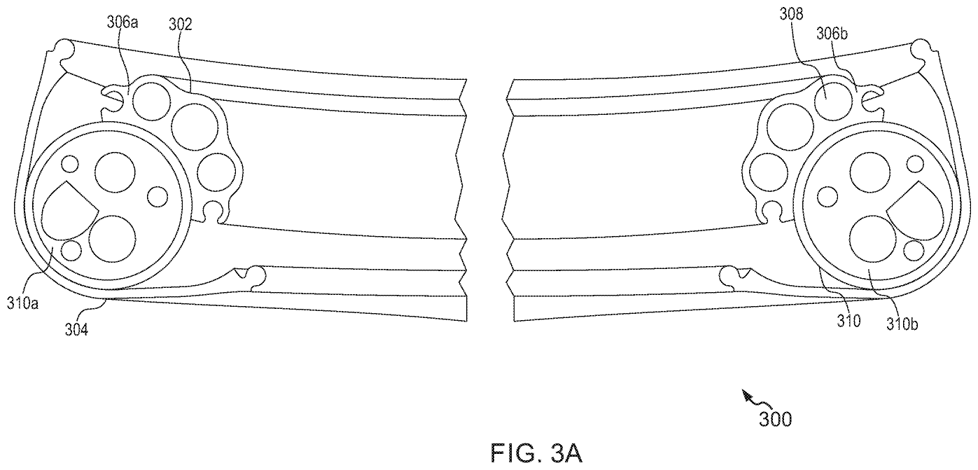

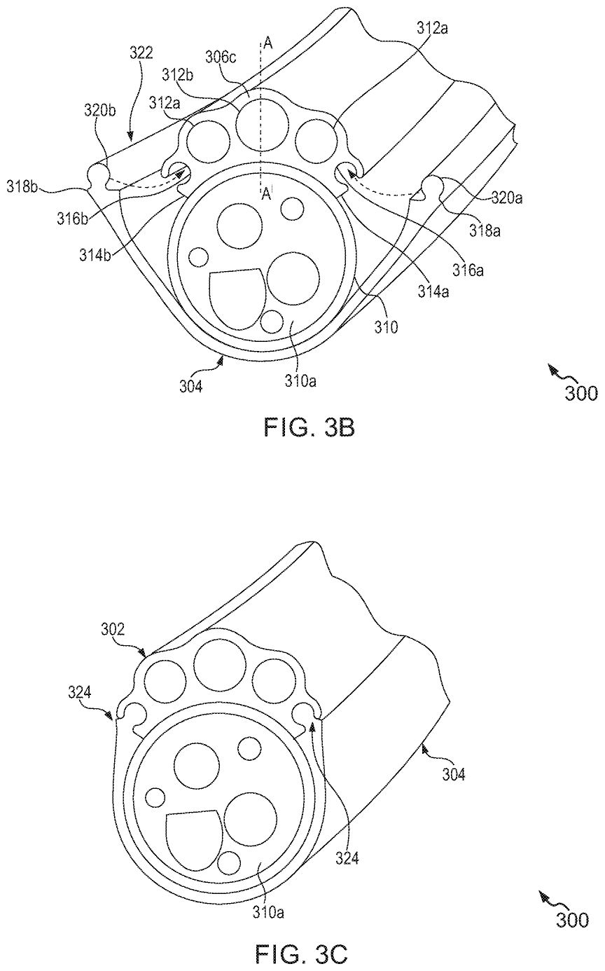

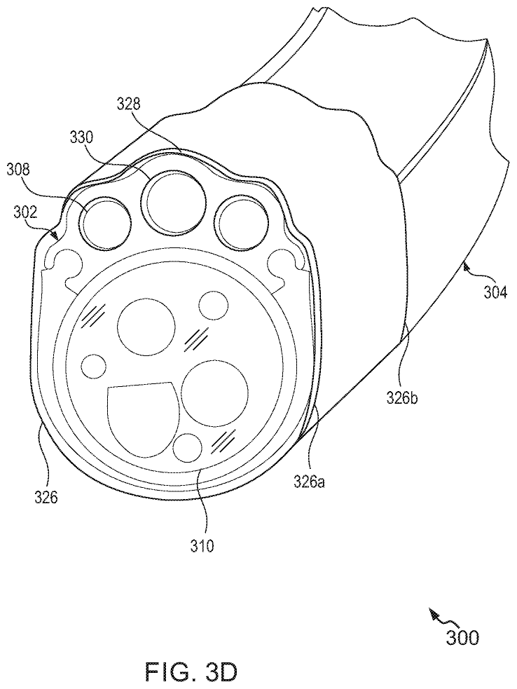

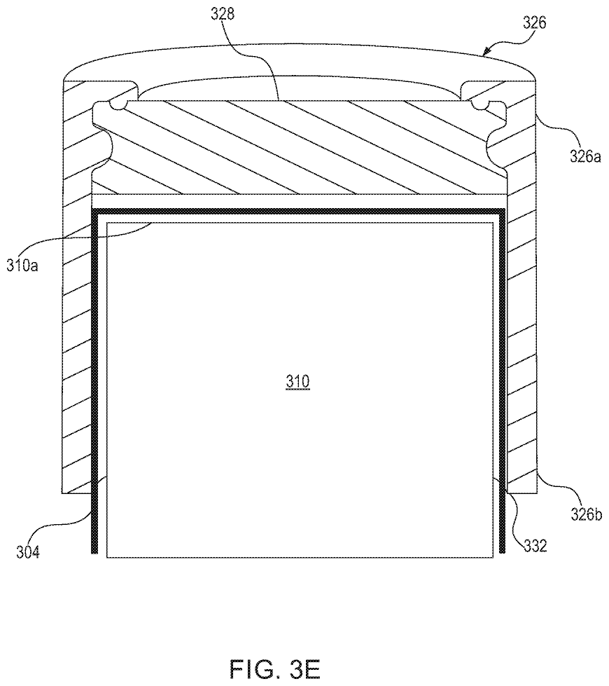

FIG. 3A illustrates a schematic view of a disposable sheath 300 for a medical device, in accordance with an example embodiment of the present disclosure. The disposable sheath 300 (hereinafter interchangeably referred to as “sheath 300”) is configured to encase a medical device, such as an endoscope 310. The endoscope 310 is an example of the endoscope 110 of FIG. 1A. It should be appreciated that a distal end 310a of the endoscope 310 may be located within the internal organ and a proximal end 310b of the endoscope 310 may be located outside the subject.

Similar to the sheath 100 of FIG. 1A, the sheath 300 includes an elongated conduit 302 (hereinafter interchangeably referred to as “conduit 302”) and a shield body member 304. The elongated conduit 302 includes a proximal portion 306a and a distal portion 306b. The elongated conduit 302 is configured with a plurality of hollow ports 308 extending from the proximal portion 306a to the distal portion 306b. In other words, the hollow ports 308 are configured along a length of the elongated conduit 302. The hollow ports 308 include a first hollow port 312a and a second hollow port 312b (as shown in FIG. 3B). The elongated conduit 302 including the first hollow port 312a and the second hollow port 312b is symmetric to an axis A-A′ of the conduit 302 (as shown in FIG. 3B). The first hollow port 312a is configured to allow passage for fluids (i.e. air or water) during the endoscopy. The second hollow port 312b corresponds to a biopsy channel. Further, the axis A-A′ may be a central axis of the elongated conduit 302. For illustrative purposes, only 3 hollow ports are illustrated in the elongated conduit 302, and they can be in any other number depending on design feasibility and requirement.

The conduit 302 includes a first end 314a and a second end 314b. It should be noted that the elongated conduit 302 towards the first end 314a (i.e. towards the right side) and the second end 314b (i.e. towards the left side) from the central axis A-A′ conforms to a stepped profile (as shown in FIG. 3B). The elongated conduit 302 may be made of flexible materials such as, but not limited to, silicon, thermoplastic elastomers (TPE) and the like.

Further, the conduit 302 includes a first socket member 316a and a second socket member 316b configured at the first end 314a and the second end 314b, respectively. The socket members 316a, 316b extend from the proximal portion 306a to the distal portion 306b (as shown in FIG. 3A). As an example, the socket members 316a, 316b may be configured to be a curved groove structure. It is noted that the first hollow port 312a is configured proximate to the first socket member 316a and the second socket member 316b, and the second hollow port 312b is configured at a central portion 306c of the conduit 302.

Further, the shield body member 304 includes a primary end 318a and a secondary end 318b. The primary end 318a and the secondary end 318b include a first engagement member 320a and a second engagement member 320b, respectively. The shield body member 304 may be configured with a predefined thickness (i.e. uniform thickness). In one configuration, the thickness of the shield body member 304 may be in the range of about 5 mm to 1 cm, as per feasibility and requirements. The shield body member 304 may be configured with flexible materials such as, but not limited to, thermoplastic elastomers (TPE), polymers, silicon and the like. In one configuration, an outer surface of the shield body member 304 and the conduit 302 may be provided with a biocompatible lubricant such as TriboSys™ PFPE (perfluoropolyether) or any other biocompatible lubricants, as per the design feasibility and requirements. The lubricant eases maneuvering of the endoscope 310 encased within the sheath 300 for accessing the internal organ.

The sheath 300 is configured to operate between a disengaged position (see, 322 of FIG. 3B) and an engaged position (see, 324 of FIG. 3C) for encasing the endoscope device 310. More specifically, the conduit 302 is rested on a portion of an outer surface of the endoscope 310. It should be noted that the socket members 316a, 316b are oriented away from the endoscope 310 when the conduit 302 is rested over the endoscope 310 (as shown in FIG. 3B). Thereafter, the shield body member 304 is wrapped around the remaining portion of the endoscope 310 and the engagement members 320a, 320b are positioned proximate to the socket members 316a, 316b, respectively (as shown in FIG. 3B). It is noted that the conduit 302 and the shield body member 304 are configured as separate components of the sheath 300. In other words, the shield body member 304 and the conduit 302 are in a two-part form or a spilt configuration in the disengaged position 322. Thereafter, the shield body member 304 is operated to the engaged position 324 by wrapping the shield body member 304 around the endoscope 310 (as shown in FIG. 3C), thereby encasing the endoscope 310. More specifically, upon positioning the conduit 302 over the endoscope 310, the first engagement member 320a and the second engagement member 320b are sealingly coupled with the first socket member 316a and the second socket member 316b, respectively, upon wrapping the shield body member 304 over the endoscope 310 (as shown in FIG. 3C). The first engagement member 320a and the second engagement member 320b sealingly coupled with the first socket member 316a and the second socket member 316b, respectively, form a unitary structure of the sheath 300.

Further, the engagement members 320a, 320b may be configured with a biocompatible adhesive for sealingly coupling with the socket members 316a, 316b. The biocompatible adhesive may provide temporary adhesion. The adhesion provides adequate coupling and further prevents deformation/misalignment of the shield body member 304 while maneuvering the endoscope 310 wrapped with the sheath 300 in the internal organ. Thus, the sheath 300 may be disengaged (i.e. operated to the disengaged position 322), upon completion of the endoscopy for disposal. In one scenario, the sheath 300 may be washed and re-used.

In an embodiment, the conduit 302 and the shield body member 304 may be sealingly coupled (i.e. operated to the engaged position 324), thereby forming an opening for allowing the endoscope 310 to be inserted therethrough.

Further, an endoscope cap 326 is releasably engaged with a portion of the elongated conduit 302 and the shield body member 304 covering the distal end 310a of the endoscope 310 (as shown in FIG. 3D). The configuration of the endoscope cap 326 is similar to that of the endoscope cap 124 of FIGS. 1D and 1E.

The endoscope cap 326 includes a transparent lens 328 (as shown in FIG. 3D). The transparent lens 328 is disposed at a front portion 326a of the endoscope cap 326, thereby forming a closed structure at the front portion 326a of the endoscope cap 326 (as shown in FIGS. 3D and 3E). The transparent lens 328 is configured with a plurality of orifices 330. The orifices 330 are positioned coaxially with the hollow ports 308 when the endoscope cap 326 is releasably engaged with the portion of the elongated conduit 302 and the shield body member 304 encompassing the distal end 310a of the endoscope 310 (as shown in FIG. 3D). The endoscope cap 326 further includes an aperture 332 configured at a rear portion 326b of the endoscope cap 326 (as shown in FIG. 3E). The aperture 332 is configured to receive the portion of the conduit 302 and the shield body member 304 encompassing the distal end 310a of the endoscope 310 (as shown in FIG. 3E). The endoscope cap 326 conforms to a shape of the elongated conduit 302 and the shield body member 304 encasing the endoscope 310 (as shown in FIG. 3D). This enables the endoscope cap 326 to be snuggly mounted to the portion of the elongated conduit 302 and the shield body member 304 encasing the distal end 310a of the endoscope 310. This prevents misalignment or deformation of the endoscope cap 326 during the endoscopy. In an embodiment, the biocompatible adhesive may be used for temporary sealing of the endoscope cap 326 to the conduit 302 and the shield body member 304 covering the endoscope 310.

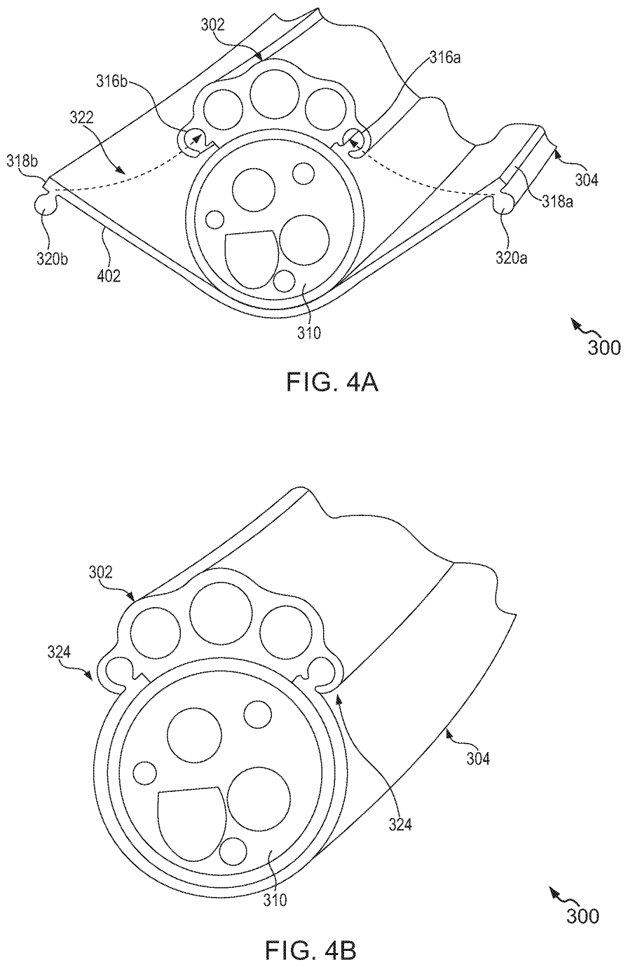

Referring to FIG. 4A, the first engagement member 320a and the second engagement member 320b may be configured on an exposed surface 402 of the shield body member 304. The engagement members 320a, 320b configured on the exposed surface 402 are located proximate to the primary end 318a and the secondary end 318b (as shown in FIG. 4A). In this configuration, the first socket member 316a and the second socket member 316b may be configured at a different orientation to receive the engagement members 320a, 320b. More specifically, the socket members 316a, 316b may be oriented towards the endoscope 310 when the conduit 302 is rested over the endoscope 310 (as shown in FIG. 4A). Thus, the engagement members 320a, 320b are sealingly coupled to the socket members 316a, 316b upon wrapping the shield body member 304 to the endoscope 310 (as shown in FIG. 4B). In this configuration, the endoscope cap 326 is releasably engaged with the portion of the conduit 302 and the shield body member 304 covering the distal end 310a of the endoscope 310 as explained above.

FIG. 5 illustrates a schematic representation of a sheath 500 encasing an endoscope 502 inserted to an internal organ of a subject, in one exemplary embodiment of the present disclosure. The sheath 500 is an example of the sheath 100 and the sheath 300 as described above. Further, the endoscope 502 is similar to the endoscope 110 and the endoscope 310 as described above. The sheath 500 is configured to encase the endoscope 502 as explained above. Upon encasing the endoscope 502, the sheath 500 along with the endoscope 502 is inserted by the medical practitioner into an internal organ 506 of a subject 504 for visual inspection. As an example, the internal organ 506 may be digestive tract of the subject 504. The sheath 500 is configured to protect the endoscope 502 from tissue fluids, thus preventing contamination of the endoscope 502. Further, the sheath 500 may provide maneuverability of the endoscope 502 for accessing the internal organ 506 during the endoscopy. The endoscope 502 encased within the sheath 500 may be connected to a control body 508. The medical practitioner may provide fluid and vacuum into the internal organ 506 through the control body 508 during the endoscopy for enhancing visualization of the internal organ 506.

Various embodiments of the disclosure, as discussed above, may be practiced with steps and/or operations in a different order, and/or with hardware elements in configurations, which are different than those which, are disclosed. Therefore, although the disclosure has been described based upon these exemplary embodiments, it is noted that certain modifications, variations, and alternative constructions may be apparent and well within the spirit and scope of the disclosure.

Although various exemplary embodiments of the disclosure are described herein in a language specific to structural features and/or methodological acts, the subject matter defined in the appended claims is not necessarily limited to the specific features or acts described above. Rather, the specific features and acts described above are disclosed as exemplary forms of implementing the claims.

What is claimed is:

1. A disposable sheath for an endoscope device, comprising:
an elongated conduit comprising a first end and a second end, the elongated conduit configured with a plurality of hollow ports extending from a proximal portion to a distal portion of the elongated conduit;
a shield body member comprising a first flap member and a second flap member extending from the first end and the second end, respectively, wherein the first flap member and the second flap member are configured along a length of the elongated conduit,
wherein the first flap member and the second flap member are operated between a closed position and an open position for encasing the endoscope device for performing an endoscopy; and
an endoscope cap adapted to releasably engage with a portion of the elongated conduit and the shield body member encompassing a distal end of the endo scope device.

2. The disposable sheath as claimed in claim 1, wherein,
in the open position, the elongated conduit is positioned over the endoscope device and a distal end of each of the first and second flap members is positioned apart from each other for allowing a user to wrap the sheath around the endoscope device; and
in the closed position, the distal end of each of the first and second flap members is sealingly coupled with each other by wrapping the first flap member and the second flap member around the endoscope device, thereby encasing the endoscope device.

3. The disposable sheath as claimed in claim 2, wherein the distal end of each of the first flap member and the second flap member is configured with a biocompatible adhesive for sealingly coupling the distal end of each of the first flap member and the second flap member.

4. The disposable sheath as claimed in claim 2, wherein the distal end of each of the first flap member and the second flap member is configured with a first taper surface and a second taper surface, respectively.

5. The disposable sheath as claimed in claim 4, wherein, in the closed position, the first taper surface is sealingly coupled to the second taper surface by overlapping the first taper surface over the second taper surface upon wrapping the first flap member and the second flap member around the endoscope device.

6. The disposable sheath as claimed in claim 5, wherein the first taper surface corresponds to an inner taper and the second taper surface corresponds to an outer taper.

7. The disposable sheath as claimed in claim 1, wherein the endoscope cap comprises a transparent lens configured with a plurality of orifices, the transparent lens disposed at a front portion of the endoscope cap, thereby forming a closed structure at the front portion of the endoscope cap, the transparent lens allows visualization of an internal organ of a subject during the endoscopy.

8. The disposable sheath as claimed in claim 1, wherein the endoscope cap comprises an aperture configured at a rear portion of the endoscope cap, the aperture configured to receive the portion of the elongated conduit and the shield body member encompassing the distal end of the endoscope device.

9. The disposable sheath as claimed in claim 8, wherein the endoscope cap conforms to a shape of the elongated conduit and the shield body member encasing the endoscope device, thereby enabling the endoscope cap to be snuggly mounted to the portion of the elongated conduit and the shield body member covering the distal end of the endoscope device.

10. The disposable sheath as claimed in claim 1, wherein the plurality of hollow ports comprises:
a first hollow port located proximate to the first end and the second end of the elongated conduit, the first hollow port configured to allow passage of fluids therethrough; and
a second hollow port located at a central portion of the elongated conduit, wherein the second hollow port corresponds to a biopsy channel and configured to allow medical instruments therethrough.

11. The disposable sheath as claimed in claim 1, wherein the elongated conduit, and the shield body member are made of flexible materials.

12. The disposable sheath as claimed in claim 1, wherein the elongated conduit towards the first end and the second end from a central axis of the elongated conduit conforms to a stepped profile.

13. A disposable sheath for an endoscope device, comprising:
an elongated conduit, comprising:
a plurality of hollow ports extending from a proximal portion to a distal portion of the elongated conduit, and
a first socket member and a second socket member configured at a first end and a second end of the elongated conduit respectively, the first socket member and the second socket member extending from the proximal portion to the distal portion;
a shield body member comprising a primary end and a secondary end, the primary end and the secondary end are configured with a first engagement member and a second engagement member, respectively, wherein the shield body member is configured to operate between an engaged position and a disengaged position for encasing the endoscope device; and
an endoscope cap adapted to releasably engage with a portion of the elongated conduit and the shield body member encompassing a distal end of the endoscope device.

14. The disposable sheath as claimed in claim 13, wherein,
in the engaged position, the elongated conduit is positioned over the endoscope device and the first engagement member and the second engagement member are sealingly coupled with the first socket member and the second socket member, respectively, upon wrapping the shield body member around the endoscope device; and
in the disengaged position, the first engagement member and the second engagement member are disengaged from the first socket member and the second socket member, respectively, thereby splitting the shield body member and the elongated conduit.

15. The disposable sheath as claimed in claim 13, wherein the first engagement member and the second engagement member sealingly coupled with the first socket member and the second socket member, respectively, form a unitary structure of the sheath.

16. The disposable sheath as claimed in claim 15, wherein the first engagement member and the second engagement member are configured with a biocompatible adhesive for sealingly coupling with the first socket member and the second socket member, respectively.

17. The disposable sheath as claimed in claim 13, wherein the first engagement member and the second engagement member are configured on an exposed surface and located proximate to the primary end and the secondary end of the shield body member respectively.

18. The disposable sheath as claimed in claim 13, wherein the elongated conduit towards the first end and the second end from a central axis of the elongated conduit conforms to a stepped profile.

19. The disposable sheath as claimed in claim 13, wherein the endoscope cap comprises a transparent lens configured with a plurality of orifices, the transparent lens disposed at a front portion of the endoscope cap, thereby forming a closed structure at the front portion of the endoscope cap, the transparent lens allows visualization of an internal organ of a subject during the endoscopy.

20. The disposable sheath as claimed in claim 13, wherein the endoscope cap comprises an aperture configured at a rear portion of the endoscope cap, the aperture configured to receive the portion of the elongated conduit and the shield body member encompassing the distal end of the endoscope device.

21. The disposable sheath as claimed in claim 20, wherein the endoscope cap conforms to a shape of the elongated conduit and the shield body member encasing the endoscope device, thereby enabling a snug mount of the endoscope cap to the portion of the elongated conduit and the shield body member covering the distal end of the endoscope device.

22. The disposable sheath as claimed in claim 13, wherein the plurality of hollow ports comprises:
a first hollow port located proximate to the first socket member and the second socket member, the first hollow port configured to allow passage of fluids therethrough; and
a second hollow port located at a central portion of the elongated conduit, the second hollow port corresponds to a biopsy channel.

23. The disposable sheath as claimed in claim 13, wherein the elongated conduit and the shield body member are made of flexible materials.
